(19) United States
(12) Patent Application Publication
(10) Pub. No.: US 2008/0107728 A1
(43) Pub. Date: May 8, 2008

(54) FORMULA AND METHOD FOR MAKING RELIEF LOZENGES AND METHOD BY WHICH INGREDIENTS IN LOZENGES ARE DELIVERED TO USER

(76) Inventor: Chris Calpito, Imperial Beach, CA (US)

Correspondence Address: BARNES & THORNBURG LLP, 600 ONE SUMMIT SQUARE, FORT WAYNE, IN, 46802, US

(21) Appl. No.: 11/934,444

(22) Filed: Nov. 2, 2007

Related U.S. Application Data
(60) Provisional application No. 60/856,438, filed on Nov. 4, 2006.

(51) Int. Cl. A61K 9/20; A61K 36/25; A61P 43/00

(52) U.S. Cl. 424/464; 424/728

(57) ABSTRACT

A lozenge or tablet which has a lozenge or tablet base, eleuthero, St. John's Wort, Valerian, and Passion flower.

### RELATED APPLICATIONS

The present application is related to and claims priority to U.S. Provisional Patent Application, Ser. No. 60/856,438, filed on Nov. 4, 2006, entitled Formula and Method for Making Relief Lozenges and Method by Which Ingredients are Delivered to the User. To the extent not included below, the subject matter disclosed in the referenced application is hereby expressly incorporated into the present application.

### BACKGROUND AND SUMMARY

The present disclosure is directed to lozenges or tablets that include a herbal mixture, as well as a method of making the same.

Lozenges and tablets are known for use in relieving sore throats and cold symptoms. Certain herbs are known to have certain energy enhancing or sedative properties. For example, eleuthero (Eleutherococcus senticosus), also known as ginseng, is believed to be a building herb for exhaustion and depletion of energy from stress and burnout. It may increase the ability to handle stress and increases overall energy in times of fatigue and debility; St. John's Wort (Hypericum perforatum) is believed to have soothing and mood enhancing properties, and may help improve the irritability and stress associated with not smoking, for example; Valerian (Valeriana officianlis) comes from the Latin word “valere” which means “to be in good health” and is believed to be a sedative and relaxant for unwinding; and Passion flower (Passiflorae herba) is believed to be a sedative, mood elevating, anxiolytic, and antispasmodic.

Accordingly, an illustrative embodiment of the present disclosure provides a lozenge or tablet comprising: a lozenge base, eleuthero, St. John's Wort, Valerian, and Passion flower. Use of the word lozenge should be interpreted to include a lozenge or a tablet, as both are designed to dissolve in the mouth, rather than chewed and swallowed like food, or swallowed whole like a pill.

In the above and other illustrative embodiments, the lozenge or tablet may further comprise: a formulation comprising about 16 to 17 percent eleuthero, 1.7-1.8 percent St. John's Wort, about 0.03 percent Valerian, and about 0.001 percent Passion flower; the lozenge base having a formulation comprising about 40-41 percent sugar, about 38-39 percent corn syrup, about 0.37 percent peppermint oil; the lozenge being made from a process whereby the eleuthero, St. John's Wort, Valerian and Passion flower are heated before the lozenge is formed; wherein the lozenge is made from a cold-press process whereby the eleuthero, St. John's Wort, Valerian and Passion flower are not heated prior to forming the lozenge; the lozenge base having a formulation comprising sugar, corn syrup, and peppermint oil.

Another illustrative embodiment includes a method of adding constituents to the manufacture of a lozenge, the method comprising the steps of: providing a formulation of about 16 to 17 percent eleuthero, 1.7-1.8 percent St. John's Wort, about 0.03 percent Valerian, and about 0.001 percent Passion flower; and adding the eleuthero, St. John's Wort, Valerian, and Passion flower to a lozenge formulation at a temperature between about 180 to about 190 degrees Fahrenheit. Another illustrative embodiment includes the step of adding the eleuthero, St. John's Wort, Valerian, and Passion flower to the lozenge formulation at a temperature between about 180 to about 190 degrees Fahrenheit after the lozenge formulation was heated to a temperature of about 260 degrees Fahrenheit.

Additional features and advantages of the lozenge or tablet, as well as their manufacture, will become apparent to those skilled in the art upon consideration of the following detailed description of the illustrated embodiment exemplifying the best mode of lozenge or tablet, as well as their manufacture as presently perceived.

### DETAILED DESCRIPTION

The present disclosure is directed to a herbal mixture of Valerian, St. John's Wort, Ginseng, and Passion flower for human consumption. The combination of a known energy enhancer with known sedatives produces a combination believed to create a feeling of wellness and relaxation from stress and anxiety.

The combination may be added to a lozenge or tablet to deliver the herbs into the body. This delivery system is employed because rather than swallowing a capsule/pill that dissolves in the stomach, the lozenge or tablet dissolves in the mouth. The saliva which receives the liquid excreted from the lozenge is absorbed into the bloodstream via the tongue and throat membranes. In addition, the “sucking” action on the lozenge occupies the mouth area, rather than grinding teeth, tight jaws, and neck muscles. This can precipitate a more relaxed state of mind.

The method of manufacturing the lozenge is the same as the conventional method known to those skilled in the art. Thus, repeating the known process is not necessary. This method is modified, however, by adding a herbal mixture of Valerian, St. John's Wort, Ginseng, and Passion flower to the liquid lozenge mixture at a temperature between about 180 to about 190 degrees Fahrenheit. In an embodiment, the herbal mixture is added to the liquid lozenge mixture after it reaches the “crack” stage (about 260 degrees Fahrenheit). It is believed this procedure maintains the effectiveness and integrity of the herbs during human consumption.

An alternative method of making the relief lozenges produces a “pressed tablet.” The pressed tablet is made by a “cold” process in which no heat is used. The same formula is used which includes the herbal mixture (Valerian, St. John's Wort, Ginseng, and Passion flower). For the pressed tablets, their strength in compression is within specified limits. Their thickness is also within specified limits. The pressed tablets disintegrate in a liquid at about 37.7 degrees Celsius (i.e., body temperature).

Hence, these lozenges or tablets keep the effectiveness and integrity of the herbs, so that during human consumption a feeling of wellness and calmness within the mind and body is experienced.

This disclosure is further illustrated, but is not intended to be limited, by the following illustrative formulation and processing for 8,000 4.5 gram lozenges:

| Component Description | Ingredient Amount |
|---|---|
| Liquid sucrose | 60 lbs |
| Corn syrup 82 Brix | 49 lbs |
| Ginseng flavor concentrate | 1.40 lbs |
| Passion flower | 45 gm |
| “Blend Mix” (St. John's Wort/Valerian) | 2.5 lbs (836 gm) |
| Peppermint oil | 45 gm |
| Horehound extract | 45 gm |

Note: Ginseng concentrate “blend mix” and Passion flower can be added at 260 degrees Fahrenheit; horehound extract is added at 250 degrees Fahrenheit. The process may include a vacuum at 260 degrees Fahrenheit, 1% to 2% at 5 minutes moisture loss which makes the lozenge drier and less tacky, thus producing a longer shelf life (approximately 2 years) from date of production/manufacturing.

The following formulation is for an approximate 4 gram lozenge or pressed tablet:

| Weight | | Percentage per |
|---|---|---|
| Grams | Ingredients | Lozenge/Pressed Tablet |
| 0.9 | Ginseng (Eleuthero) FE | 16.66 |
| | (fluid extract) | |
| 0.096 | St. John's Wort FE (fluid extract) | 1.77 |
| 0.002 | Valerian FE (fluid extract) | 0.03 |
| 0.0005 | Passion flower FE (fluid extract) | 0.001 |
| 0.07 | water | 1.29 |
| 2.2 | sugars/syrup | 40.7 |
| 2.1 | corn syrup | 38.8 |
| 0.02 | peppermint oil | .37 |

Liquid formulation for manufacturing and production is about 5.408 grams (100%). For the lozenge the process includes heating the formulation to about 300 degrees Fahrenheit, then apply a cool vacuum, and set into a desired mold shape (round, oval, specific size etc.). For a pressed tablet using a cold press method (i.e., no heat) the above formulation is placed in a cold-pressed tablet machine and pressed into the desired shape (round, oval, etc.).

Although the present disclosure has been described with reference to particular means, materials, and embodiments, from the foregoing description one skilled in the art can easily ascertain the essential characteristics of the present disclosure and various changes and modifications may be made to adapt the various uses and characteristics without departing from the spirit and scope of the present invention as set forth in the following claims.

What is claimed is:

1. A lozenge comprising: a lozenge base, eleuthero, St. John's Wort, Valerian, and Passion flower.

2. The lozenge of claim 1, further comprising a formulation of about 16 to 17 percent eleuthero, 1.7-1.8 percent St. John's Wort, about 0.03 percent Valerian, and about 0.001 percent Passion flower.

3. The lozenge of claim 1, wherein the lozenge base has a formulation comprising about 40-41 percent sugar, about 38-39 percent corn syrup, about 0.37 percent peppermint oil.

4. The lozenge of claim 1, wherein the lozenge is made from a heated process.

5. The lozenge of claim 1, wherein the lozenge is made from a cold-press process.

6. The lozenge of claim 1, wherein the lozenge base has a formulation comprising sugar, corn syrup, and peppermint oil.

7. A tablet comprising: a base, eleuthero, St. John's Wort, Valerian, and Passion flower.

8. The lozenge of claim 7, further comprising a formulation of about 16 to 17 percent eleuthero, 1.7-1.8 percent St. Johns Wort, about 0.03 percent Valerian, and about 0.001 percent Passion flower.

9. A method of adding constituents to the manufacture of a lozenge, the method comprising the steps of: providing a formulation of about 16 to 17 percent eleuthero, 1.7-1.8 percent St. John's Wort, about 0.03 percent Valerian, and about 0.001 percent Passion flower; and adding the eleuthero, St. John's Wort, Valerian, and Passion flower to a lozenge formulation at a temperature between about 180 to about 190 degrees Fahrenheit.

10. The method of adding constituents to the manufacture of the lozenge of claim 9, further comprising the step of adding the eleuthero, St. John's Wort, Valerian, and Passion flower to the lozenge formulation at a temperature between about 180 to about 190 degrees Fahrenheit after the lozenge formulation was heated to a temperature of about 260 degrees Fahrenheit.
